(19) United States
(12) Patent Application Publication
(10) Pub. No.: US 2012/0129259 A1
(43) Pub. Date: May 24, 2012

(54) METHOD FOR PRODUCING A COATED CELL CULTURE CARRIER

(75) Inventors: Jürgen Köcher, Langenfeld (DE); Theresia Klose, Dresden (DE); Tilo Pompe, Dresden (DE); Philipp Seib, Dresden (DE); Carsten Werner, Dresden (DE)

(73) Assignee: BAYER MATERIALSCIENCE AG, Leverkusen (DE)

(21) Appl. No.: 13/321,844

(22) PCT Filed: May 18, 2010
(86) PCT No.: PCT/EP10/03022

§ 371 Date: Feb. 13, 2012

(30) Foreign Application Priority Data
May 27, 2009 (EP) 09007050.9

(51) Int. Cl. C12N 5/071; B05D 5/00; B05D 3/00; C12N 5/0775

(52) U.S. Cl. 435/402; 427/385.5

(57) ABSTRACT

The present invention relates to a method for producing a coated cell culture carrier, wherein a solution comprising a polyurethane urea is applied to a cell carrier and dried. The polyurethane urea is produced beforehand by converting at least one polycarbonate polyol component, at least one polyisocyanate component, and at least one diamino component. The invention further relates to a cell culture carrier obtained according to the method and the use thereof for in-vitro cultivation of stem cells, particularly for cultivating mesenchymal stem cells.

The present invention relates to a method for producing a coated cell culture carrier in which a polyurethane-urea-containing solution is applied to a cell carrier and dried. The invention further relates to a cell culture carrier obtainable by the method and the use thereof for in-vitro culturing of stem cells, in particular for culturing mesenchymal stem cells.

Mesenchymal stem cells are capable of either multiplying or differentiating into different cell types such as osteoblasts, chondrocytes or adipocytes (A. I. Caplan and J. E. Dennis, J. Cell Biochem. 98, 2006, 1076-1084). The multipotency of mesenchymal stem cells paired with the easy isolability from adult donors makes these stem cells an ideal source for cells for tissue engineering (D. P. Lennon and A. I. Caplan, Exp. Hematol. 34, 2006, 1604-1605). Examples of such uses are regeneration of cartilage or bones or for therapeutic measures for treating stroke or heart infarction (A. I. Caplan and J. E. Dennis, J. Cell Biochem. 98, 2006, 1076-1084). On account of the low concentration of these mesenchymal stem cells in human bone marrow, it is necessary to culture and multiply these stem cells in vitro before clinical use (A. I. Caplan and J. E. Dennis, J. Cell Biochem. 98, 2006, 1076-1084; D. L. Jones and A. J. Wagers, Nat. Rev. Mol. Cell Biol. 9, 2008, 11-21). However, in this case hitherto very frequently loss of the differentiation potential and thus reduced therapeutic benefit frequently occurs (S. J. Morrison and A. C. Spradling, Cell 132, 2008, 598-611). During relatively long periods of culturing, mesenchymal stem cells frequently show properties of osteoblasts and have therefore already lost differentiation potential (Banfi et al., Exp Hematol 28, 2000, 707; Baxter et al., Stem Cells 22, 2004 675; Wagner et al., PLoS ONE 3, 2008, e2218).

Quite in general, strategies are desired in order to be able to culture mesenchymal stem cells in-vitro. Culturing here should proceed without premature differentiation of the cells and therefore without loss of the potential of the stem cells.

An established method for achieving this aim is the use of proteins of the extracellular matrix. This procedure is described, for example, in the publications X.-D. Chen et al., Journal of Bone and Mineral Research 22 (12), 2007, 1943-1956, and T. Matsubara et al., Biochemical and Biophysical Research Communications 313 (2004), 503-508. The protein mixtures used here are applied to cell culture carriers made of plastic. On the coated cell culture carriers, mesenchymal stem cells can be multiplied with lower loss of differentiation potential compared with uncoated cell culture carriers.

The multiplication of stem cells with simultaneous prevention of premature differentiation of these stem cells is also achieved in the prior art by the targeted addition of biological factors. For instance, Ansellem et al., Nature Medicine 9 (11), 2003, 1423, describe the use of modulators such as “sonic hedgehog” or Wnt proteins for preventing the differentiation of stem cells in an in-vitro culture. Patent applications WO 2006/006171 and WO 2006/030442 and also the publication PNAS 103 (2006), 11707 describes similar concepts.

The abovedescribed concepts require additional materials as modulators or as a surface layer. However, these materials are difficult to isolate, since they are natural proteins. Therefore, alternative strategies that likewise make possible multiplication with simultaneous prevention of differentiation are desirable.

A relatively new approach for the field of activity of tissue engineering is the specific design of cell culture carriers themselves for differentiation of stem cells in situ (S. Neuss et al., Biomaterials 29, 2008, 302-313). Neuss et al. study a library of different natural or artificial polymers for this purpose, wherein here, no proteins are used for supporting the stem cell culturing.

In J. M. Curran et al., Biomaterials 27, 2006, 4783-4793, the principle that the surface quality of a substrate can affect the differentiation of mesenchymal stem cells is described. It could be demonstrated on modified glass surfaces that different surface modifications affect the differentiation of mesenchymal stem cells differently. Amino- and thiol-containing glass surfaces promoted the differentiation of mesenchymal stem cells, whereas the control glass and a methyl-modified glass maintained the phenotype. However, the process of modifying a glass surface by chemical reagents is complex. Furthermore, despite everything, when an inducing agent is added, premature differentiation of the cells on the modified surface occurs.

An interesting polymer class for medical technology applications and for tissue engineering is the class of polyurethanes. These have a great potential for varying the structure and therefore for setting defined properties. Polyurethanes as matrices for stem cells are regularly used in tissue engineering. Examples thereof are described in various publications.

H. L. Pritchard et al., Biomaterials 28 (2007), 936-946 describe colonization studies of stem cells from fat cells on various substrates, inter alia, also on polyurethanes. In the case of the polyurethane Pellethane used, the colonization densities on pure material are very poor (<10%). Only further measures such as covering with fibronectin and plasma activation lead to sufficient colonization density. These measures, however, mean additional working steps and costs.

C. Alperin et al., Biomaterials 26 (2005), 7377-7386 describe culturing cardiomyocytes on polyurethanes by colonization with embryonal stem cells from mice. The stem cells are differentiated to form cardiomyocytes in a targeted manner within 9 days. Preventing the differentiation of the stem cells, in contrast, is not a subject matter of the publication.

Nieponice et al., Biomaterials 29 (2008), 825-833 describe colonization of stem cells from muscles on biodegradable polyurethane for producing implants for cardiovascular applications. The colonization proceeds on the pure polyurethane without further additives. Using the method described, the cells may be cultured on the carrier for 7 days without premature differentiation. However, the use of a complex vacuum colonization technique is necessary in order to obtain sufficient colonization density.

In the prior art, no method which may be carried out simply is known for producing a coated cell culture carrier that can readily be colonized with a sufficiently high density of stem cells, that makes possible rapid multiplication of the stem cells and that prevents premature differentiation of the stem cells during multiplication thereof.

It was therefore the object of the present invention to provide a method of the type indicated at the outset, by means of which a cell culture carrier can be obtained which equally meets the abovementioned requirements.

This object is achieved according to the invention in that the polyurethane urea contained in the solution is produced by reacting at least one polycarbonate polyol component, at least one polyisocyanate component and at least one diamine component.

A cell culture carrier produced by the method according to the invention can rapidly and simply, i.e., in particular, without the necessity of using complex techniques, be colonized with a sufficient density of stem cells. During the subsequent rapid multiplication of the stem cells on the cell culture carrier, premature unwanted differentiation of the stem cells does not occur. This effect is achieved solely by the polyurethane urea coating, i.e. without the additional use of proteins or further natural substances in the coating of the cell culture carrier. The stein cells that are multiplied on the cell culture carrier, after removal from the carrier, still exhibit the necessary differentiation potential and can be used appropriately, for example for tissue engineering.

Polyurethane ureas, in the context of the present invention, are, in particular, polymeric compounds which have

(a) at least two urethane group-containing repeating units of the following general structure

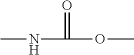

and

(b) at least one urea-group-containing repeating unit

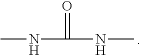

The polyurethane ureas are preferably substantially linear molecules, but can also be branched, which is less preferred, however. Substantially linear molecules is taken to mean, in the context of the present invention, slightly cross-linked systems, wherein the underlying polycarbonate polyol component has, in particular, a median hydroxyl functionality from 1.7 to 2.3, preferably from 1.8 to 2.2, and particularly preferably from 1.9 to 2.1.

In addition, the polycarbonate polyol component can have a molecular weight defined by the OH number from preferably 400 to 6000 g/mol, particularly preferably from 500 to 5000 g/mol, and especially preferably from 600 to 3000 g/mol. Such polycarbonate polyol components are obtainable, for example, by reaction of carbonic acid derivatives, such as diphenyl carbonate, dimethyl carbonate or phosgene, with polyols, preferably diols. Diols which come into consideration here are, for example, ethylene glycol, 1,2- and 1,3-propanediol, 1,3- and 1,4-butane-diol, 1,6-hexanediol, 1,8-octanediol, neopentyl glycol, 1,4-bishydroxymethylcyclohexane, 2-methyl-1,3-propanediol, 2,2,4-trimethylpentane-1,3-diol, di-, tri- or tetraethylene glycol, dipropylene glycol, polypropylene glycols, dibutylene glycol, polybutylene glycols, bisphenol A, tetrabromobisphenol A, but also lactone-modified diols.

The abovementioned polycarbonate polyols contain preferably 40 to 100% by weight of hexanediol, preferably 1,6-hexanediol and/or hexanediol derivatives, preferably those which, in addition to terminal OH groups, have ether or ester groups, e.g. products which have been obtained by reacting 1 mol of hexanediol with at least 1 mol, preferably 1 to 2 mol, of caprolactone or by etherifying hexanediol with itself to form di- or trihexylene glycol. Polyether polycarbonate diols can also be used. The hydroxyl polycarbonates should be substantially linear. However, they can optionally be slightly branched by the incorporation of polyfunctional components, in particular low-molecular-weight polyols. Polyols suitable for this purpose are, for example, glycerol, trimethylol propane, hexane-1,2,6-triol, butane-1,2,4-triol, trimethylol propane, pentaerythritol, quinitol, mannitol, sorbitol, methylglycoside or 1,3,4,6-dianhydro hexitols. Preference is given to those polycarbonates based on hexane-1,6-diol and also modifying co-diols such as, e.g. butane-1,4-diol or else c-caprolactone. In a preferred embodiment, polycarbonate polyols based on hexane-1,6-diol, butane-1,4-diol or mixtures thereof are used.

The polyurethane ureas in addition comprise units which are derived from at least one polyisocyanate component.

As polyisocyanate component, all aromatic, araliphatic, aliphatic and cycloaliphatic isocyanates having a median NCO functionality ≧1, preferably ≧2, that are known to those skilled in the art can be used individually or in any desired mixtures with one another, wherein it is irrelevant whether these were produced by phosgene or phosgene-free methods. They can also comprise iminooxadiazinedione, isocyanurate, uretdione, urethane, allophanate, biuret, urea, oxadiazine-trione, oxazolidinone, acylurea and/or carbodiimide structures. The isocyanates can be used individually or in any desired mixtures with one another.

Preferably, isocyanates from the group of aliphatic or cycloaliphatic members are used, wherein these comprise a carbon backbone (without the NCO groups contained) of 3 to 30, preferably 4 to 20 carbon atoms.

Particularly preferred compounds of the abovementioned type having aliphatically and/or cycloaliphatically bound NCO groups are, for example, bis-(isocyanatoalkyl) ethers, bis- and tris-(isocyanatoalkyl)benzenes, -toluenes, and also -xylenes, propane diisocyanates, butane diisocyanates, pentane diisocyanates, hexane diisocyanates (e.g. hexamethylene diisocyanate, HDI), heptane diisocyanates, octane diisocyanates, nonane diisocyanates (e.g. trimethyl-HDI (TMDI) generally as a mixture of the 2,4,4- and 2,2,4-isomers), nonane triisocyanates (e.g. 4-isocyanatomethyl-1,8-octane diisocyanate), decane diisocyanates, decane triisocyanates, undecane diisocyanates, undecane triisocyanates, dodecane diisocyanates, dodecane triisocyanates, 1,3- and 1,4-bis-(isocyanatomethyl)cyclohexane (H₆XDI), 3-isocyanatomethyl-3,5,5-trimethyl-cyclohexyl isocyanate (isophorone diisocyanate, IPDI), bis-(4-isocyanatocyclohexyl)methane (H₁₂MDI) or bis-(isocyanatomethyl)norbornane (NBDI).

Very particular preference is given to hexamethylene diisocyanate (HDI), trimethyl-HDI (TMDI), 2-methylpentane-1,5-diisocyanate (MPDI), isophorone diisocyanate (IPDI), 1,3- and 1,4-bis(iso-cyanatomethyl)cyclohexane (H₆XDI), bis(isocyanatomethyl)norbornane (NBDI), 3(4)-isocyanate-methyl-1-methylcyclohexyl isocyanate (IMCI) and/or 4,4′-bis-(isocyanatocyclohexyl)methane (H₁₂MDI) or mixtures of these isocyanates. Further examples are derivatives of the abovementioned diisocyanates having a uretdione, isocyanurate, urethane, allophanate, biuret, iminooxadiazinedione and/or oxadiazinetrione structure having more than two NCO groups.

The amount of polyisocyanate component in the production of the polyurethane ureas is preferably 1.0 to 5.0 mol, particularly preferably 1.0 to 4.5 mol, in particular 1.0 to 4.0 mol, based on 1 mol of the polycarbonate component.

The polyurethane ureas essential to the invention comprise units which are derived from at least one diamine and act as what are termed chain extenders.

Suitable diamine components are di- or polyamines and also hydrazides, e.g. hydrazine, ethylene-diamine, 1,2- and 1,3-diaminopropane, 1,4-diaminobutane, 1,6-diaminohexane, isophorone-diamine, isomeric mixture of 2,2,4- and 2,4,4-trimethylhexamethylenediamine, 2-methylpenta-methylenediamine, diethylenetriamine, 1,3- and 1,4-xylylenediamine, α,α,α′,α′-tetramethyl-1,3- and -1,4-xylylenediamine and 4,4′-diaminodicyclohexylmethane, dimethylethylenediamine, hydrazine, adipic acid dihydrazide, 1,4-bis(aminomethyl)cyclohexane, 4,4′-diamino-3,3′-dimethyl-dicyclohexylmethane and other (C₁-C₄)-di- and tetraalkyldicyclohexylmethanes, e.g. 4,4′-diamino-3,5-diethyl-3′,5′-diisopropyldicyclohexylmethane.

In the production of the polyurethane urea, as diamine component, low-molecular-weight diamines also come into consideration that comprise active hydrogen having different reactivity from NCO groups. These are, e.g., compounds which, in addition to a primary amino group, also comprise secondary amino groups.

Examples of such diamino components are primary and secondary amines, such as 3-amino-1-methyl aminopropane, 3-amino-1-ethylaminopropane, 3-amino-1-cyclohexylaminopropane, 3-amino-1-methylaminobutane.

According to a preferred embodiment, the diamino component comprises at least one further hydroxyl group. The diamino component here can contain both primary and secondary amines and also mixtures of both amine types. One example of a particularly preferred compound is 1,3-diamino-2-propanol.

The amount of the diamino component in the production of the polyurethane urea is preferably 0.1 to 3.0 mol, particularly preferably 0.2 to 2.8 mol, in particular 0.3 to 2.5 mol, based on 1 mol of the polycarbonate component.

In a further embodiment, in the production of the polyurethane urea, a polyol component is additionally co-reacted.

The polyol components used for the structure of the polyurethane ureas generally effect a stiffening and/or branching of the polymer chain. The molecular weight of the polyol component is preferably 62 to 500 g/mol, particularly preferably 62 to 400 g/mol, in particular 62 to 200 g/mol.

Suitable polyol components can contain aliphatic, alicyclic or aromatic groups. Those which may be mentioned here are, for example, low-molecular-weight polyol components having up to about 20 carbon atoms per molecule, such as, e.g., ethylene glycol, diethylene glycol, triethylene glycol, 1,2-propanediol, 1,3-propanediol, 1,4-butanediol, 1,3-butylene glycol, cyclohexanediol, 1,4-cyclo-hexanedimethanol, 1,6-hexanediol, neopentyl glycol, hydroquinone dihydroxyethyl ether, bisphenol A (2,2-bis(4-hydroxyphenyl)propane), hydrogenated bisphenol A (2,2-bis(4-hydroxy-cyclohexyl)propane), and also trimethylol propane, glycerol or pentaerythritol and mixtures of these and optionally other low-molecular-weight polyols. Ester diols such as, e.g., α-hydroxybutyl-ε-hydroxy-caproic acid esters, ω-hydroxyhexyl-γ-hydroxybutyric acid esters, adipic acid β-hydroxyethyl esters or terephthalic acid bis(β-hydroxyethyl)esters can also be used.

The amount of polyol component in the production of the polyurethane ureas is preferably 0.05 to 2.0 mol, particularly preferably 0.05 to 1.5 mol, in particular 0.1 to 1.0 mol, based on 1 mol of the polycarbonate component.

The reaction of the polyisocyanate component with the polycarbonate polyol component and the diamino component customarily proceeds with maintenance of a slight NCO excess compared with the reactive hydroxyl or amine compounds. At the end point of the reaction, owing to reaching a target viscosity, residues of active isocyanate still always remain. These residues must be blocked in order that reaction with large polymer chains does not take place. Such a reaction leads to three-dimensional crosslinking and to gelling of the batch. Processing of such a solution is no longer possible.

In order to block the remaining free NCO groups, they can be reacted with a blocking component. These blocking components are derived, for example, from monofunctional compounds reactive with NCO groups, such as monoamines, in particular mono-secondary amines or monoalcohols. Those which may be mentioned here are, for example, ethanol, n-butanol, ethylene glycol monobutyl ether, 2-ethylhexanol, 1-octanol, 1-dodecanol, 1-hexadecanol, methylamine, ethylamine, propylamine, butylamine, octylamine, laurylamine, stearylamine, isononyloxypropylamine, dimethylamine, diethylamine, dipropylamine, dibutylamine, N-methyl-aminopropylamine, diethyl(methyl)aminopropylamine, morpholine, piperidine and suitable substituted derivatives thereof.

Since the blocking component is primarily used for destroying the NCO excess, the amount required substantially depends on the amount of the NCO excess and cannot be specified in general.

If the residual isocyanate content was blocked in the production of the polyurethane ureas, these also comprise monomers as structural components which are in each case situated at the chain ends and terminate them.

Preferably, however, in the synthesis, no additionally added block component is used. Instead, the remaining free isocyanate groups are reacted with solvent alcohol present in very high concentration in the batch to form terminal urethanes. In this manner, the alcohol, in the course of a plurality of hours of standing or stirring the batch at room temperature, blocks the isocyanate groups still remaining.

For producing the polyurethane urea solutions, the polycarbonate polyol component, the polyisocyanate component and the diamino component are reacted with one another in a melt or in solution until all of the hydroxyl groups are consumed. Then, solvent is added.

The stoichiometry between the individual components participating in the reaction results from the abovementioned quantitative ratios.

The reaction proceeds at a temperature of preferably between 60 and 110° C., particularly preferably 75 to 110° C., in particular 90 to 110° C., wherein temperatures around 110° C. are preferred owing to the rate of the reaction. Temperatures that are still higher are likewise possible, but then in individual cases and depending on the components used, there is the risk that decomposition processes and discolorations in the resultant polymer occur.

For accelerating the isocyanate addition reaction, the catalysts known in polyurethane chemistry can be used, for example dibutyltin dilaurate. Preference, however, is given to synthesis without catalyst.

In the case of the prepolymer of isocyanate and all components having hydroxyl groups, the reaction in the melt is preferred, but there is the risk that excessive viscosities of the completely reacted mixtures will occur. In these cases, it is advisable to add solvents. However, as far as possible no more than approximately 50% by weight of solvent should be present, since otherwise the dilution markedly decreases the reaction rate.

In the case of the reaction of isocyanate and the components having hydroxyl groups, the reaction can proceed in the melt in a period of from 1 hour to 24 hours. A small addition of amounts of solvent leads to a retardation, wherein, however, the total reaction time lies within said time periods.

The sequence of addition of reaction of the individual components can differ from the above stated sequence. This can be advantageous, in particular, when the mechanical properties of the resultant coatings are to be modified. If, for example, all components having hydroxyl groups are reacted simultaneously, a mixture of hard and soft segments is formed. If, for example, the polyol is added after the polycarbonate polyol component, defined blocks are obtained which can be accompanied by other properties of the resultant coatings. The present invention is therefore not restricted to a defined sequence of addition or reaction of the individual components.

The solvent is preferably added stepwise in order not to retard the reaction unnecessarily, which would occur in the case of complete addition of the solvent, for example at the start of the reaction. In addition, in the event of a high content of solvent at the start of the reaction, a relatively low temperature is obligatory, which is at least co-determined by the type of the solvent. This also leads to a retardation of the reaction.

After reaching the target viscosity, the NCO residues still remaining can be blocked by a monoftinctional aliphatic amine. Preferably, the isocyanate groups still remaining are blocked by reaction with the alcohols present in the solvent mixture.

As solvents for producing the polyurethane urea solutions, all conceivable solvents and solvent mixtures come into consideration such as dimethylformamide, N-methylacetamide, tetramethylurea, N-methylpyrrolidone, aromatic solvents such as toluene, linear and cyclic esters, ethers, ketones and alcohols. Examples of esters and ketones are ethyl acetate, butyl acetate, acetone, γ-butyrolactone, methyl ethyl ketone and methyl isobutyl ketone.

Preference is given to mixtures of alcohols with toluene. Examples of alcohols which can be used together with toluene are ethanol, n-propanol, isopropanol and 1-methoxy-2-propanol.

Generally, in the reaction, sufficient solvent is added such that approximately 10 to 50% strength by weight solutions, preferably approximately 15 to 45% strength by weight solutions, and particularly preferably about 20 to 40% strength by weight solutions are obtained.

The solids content of the polyurethane urea solutions is generally in the range from 1 to 60% by weight, preferably from 10 to 40% by weight. For coating experiments, the polyurethane urea solutions can be diluted as desired with toluene/alcohol mixtures in order to set the thickness of the coating so as to be variable.

Any desired layer thicknesses can be achieved such as, for example, some 100 nm to some 100 μm, wherein, the context of the present invention, higher and lower thicknesses are also possible.

The polyurethane urea solutions can, in addition, contain ingredients and additives customary for the respective sought-after purpose.

Further additives such as, for example, antioxidants or pigments, can likewise be used. Furthermore, optionally, still other additives such as gripping aids, dyes, matting agents, UV stabilizers, light stabilizers, hydrophilizing agents, hydrophobicizing agents and/or free-flowing aids can be used.

The polyurethane urea solutions can additionally contain proteins. Preferably, these can be proteins of the extracellular matrix.

Coatings of the polyurethane urea solutions can be applied to the cell culture carrier by various methods. Suitable coating techniques are, for example, squeegee application, printing, transfer coating, spraying, spin coating or immersion.

It is possible to coat many types of substrates such as glass, silicon wafers, metals, ceramics and plastics. Preference is given to coating cell culture carriers made of glass, silicon wafers, plastic or metals. Metals which may be mentioned are, for example: medical stainless steel and nickel-titanium alloys. Many polymer materials are conceivable of which the cell culture carriers can be composed, for example polyamide; polystyrene; polycarbonate; polyethers; polyesters; polyvinylacetate; natural and synthetic rubbers; block copolymers of styrene and unsaturated compounds such as ethylene, butylene and isoprene; polyethylene or copolymers of polyethylene and polypropylene; silicone; polyvinyl chloride (PVC) and polyurethanes. For better adhesion of the polyurethanes according to the invention to the cell culture carrier, as substrate before application of these coating materials, still further suitable coatings can be applied. Particularly preferably, the coating is of glass or silicon wafers for producing cell culture carriers.

The advantages of the cell culture carriers produced by the method according to the invention, in particular for culturing mesenchymal stem cells, are documented by the examples cited hereinafter.

### EXAMPLES

The NCO content of the resins described in the examples and comparative examples was determined by titration as specified in DIN EN ISO 11909.

The solids contents were determined as specified in DIN EN ISO 3251. An amount of 1 g of polyurethane dispersion was dried to constant weight at 115° C. (15-20 min) by means of an infrared dryer.

The quantities stated in %, unless otherwise stated, are taken to mean % by weight and relate to the aqueous dispersion obtained.

Viscosity measurements were carried out using the Physics MCR 51 Rheometer from Firma Anton Paar GmbH, Ostfildern, Germany.

Substances and Abbreviations Used:
Desmophen C2200: Bayer MaterialScience AG, Leverkusen, Germany
   Polycarbonate polyol, OH number 56 mg KOH/g, number-average molecular weight 2000 g/mol
PolyTHF 2000 BASF AG, Ludwigshafen, Germany
   Polytetramethylene glycol polyol, OH number 56 mg KOH/g, number-average molecular weight 2000 g/mol
Cell line C3H10T0.5 Mesenchymal cell line (mouse species), Clone 8, obtained from the American Type Culture Collection (ATTC), Manassass, Va., USA, ATCC Number CCL-226
Cell line C2C12 Multipotent cell line (mouse species), obtained from the American Type Culture Collection (ATTC), Manassass, Va., USA, ATCC Number CRL-1772
Fetal calf serum (FBS) Biochrom AG, Berlin, Germany
Bone Morphogenic Protein 2 (BMP-2) Wyeth Pharma GmbH, Munster, Germany
   This is a human recombinant bone morphogenetic protein-2 (rhBMP-2) which was produced in a recombinant Chinese hamster ovary (CHO) cell line; (Dibotermin alfa)
IST+ BD Biosciences, Heidelberg, Germany
   Composition:
   Insulin 6.25 μg/ml
   Transferin 6.25 μg/ml
   Selenous acid 6.25 ng/ml
   Bovine serum albumin 1.25 mg/ml
   Linoleic acid 5.35 μg/ml
Eagle's Basal Medium (BME)(1X) Gibco/Invitrogen, Karlsruhe, Germany, Catalog No. 41010
   Liquid containing Earle's Salts, without L-glutamine
Dulbecco's Modified Eagle Medium Gibco/Invitrogen, Karlsruhe, Germany,
(D-MEM) (1X), liquid (High Glucose) Catalog No. 31966
   Product-relevant specifications:
   Glucose: high glucose content (4500 mg/L)
   Glutamine: GlutaMAX™-I
   HEPES buffer: no HEPES
   Sodium pyruvate addition: sodium pyruvate 110 mg/L
Dulbecco's Modified Eagle Medium Gibco/Invitrogen, Karlsruhe, Germany,
(D-MEM) (1X), liquid (low Glucose) Catalog No. 21885
   Product-relevant specifications:
   Glucose: high glucose content (1000 mg/L)
   Glutamine: GlutaMAX™-I
   HEPES buffer: no HEPES
   Sodium pyruvate addition: sodium pyruvate 110 mg/L
Glutamax™ 200 mM Invitrogen, Karlsruhe, Germany, Catalog No. 35050038
   Added to the Eagle's Basal Medium in order to obtain a 2 mM final concentration.
Phosphate-buffered saline (PBS) Biochrom AG, Berlin, Germany
Alamar Blue Gibco/Invitrogen, Karlsruhe, Germany

The further ingredients were DMEM (applies to the two media listed above) and BME can be seen at Invitrogen and are not manufacturer-specific.

Microtiter plates made of Tissue Culture Polystyrene (TPS) from Techno Plastic Products (TPP), Trasadingen, Switzerland were used.

### Example 1

At 110° C., 500.0 g of Desmophen C 2200, 104.6 g of isophorone diisocyanate and 126.6 g of toluene were reacted to a constant NCO content of 2.5%. The mixture was allowed to cool and was diluted with 500.0 g of toluene and 377.8 g of isopropanol. At room temperature, a solution of 34.7 g of 4,4′-diaminodicyclohexylmethane dissolved in 308.4 g of 1-methoxy-2-propanol was added. After build up of the molecular weight was completed and the desired viscosity range was reached, the mixture was allowed to stand overnight at room temperature in order to block the residual isocyanate content with isopropanol or 1-methoxy-2-propanol. This produced 1952 g of a 33.4% strength polyurethane urea solution in toluene/isopropanol/1-methoxy-2-propanol having a viscosity of 21200 mPas at 22° C.

### Example 2

At 110° C., 300.0 g of Desmophen C 2200, 11.2 g of 1,2-dodecanediol (90% pure), 104.6 g of isophorone diisocyanate and 80.0 g of toluene were reacted to a constant NCO content of 4.5%. The mixture was allowed to cool and was diluted with 350.0 g of toluene and 350.0 g of isopropanol. At room temperature, a solution of 52.5 g of 4,4′-diaminodicyclohexylmethane dissolved in 353.9 g of 1-methoxy-2-propanol was added. After build up of the molecular weight was completed and the desired viscosity range was reached, the mixture was allowed to stand overnight at room temperature in order to block the residual isocyanate content with isopropanol or 1-methoxy-2-propanol. This produced 1602 g of a 35.8% strength polyurethane urea solution in toluene/isopropanol/1-methoxy-2-propanol having a viscosity of 25000 mPas at 22° C.

### Example 3

At 110° C., 400.0 g of Desmophen C 2200, 104.6 g of isophorone diisocyanate and 126.6 g of toluene were reacted to a constant NCO content of 3.6%. The mixture was allowed to cool and was diluted with 422.4 g of toluene and 377.8 g of isopropanol. At room temperature, a solution of 22.9 g of 1,3-diamino-2-propanol dissolved in 357.7 g of 1-methoxy-2-propanol was added. After build up of the molecular weight was completed and the desired viscosity range was reached, the mixture was allowed to stand overnight at room temperature in order to block the residual isocyanate content with isopropanol or 1-methoxy-2-propanol. This produced 1812 g of 30.5% strength polyurethane urea solution in toluene/isopropanol/1-methoxy-2-propanol having a viscosity of 37000 mPas at 22° C.

### Example 4

At 110° C., 320.0 g of Desmophen C 2200, 104.6 g of isophorone diisocyanate and 126.6 g of toluene were reacted to a constant NCO content of 4.7%. The mixture was allowed to cool and was diluted with 360 g of toluene and 377.8 g of isopropanol. At room temperature, a solution of 26.4 g of 1,3-diamino-2-propanol dissolved in 369.6 g of 1-methoxy-2-propanol was added. After build up of the molecular weight was completed and the desired viscosity range was reached, the mixture was further stirred for 4 h in order to block the residual isocyanate content with isopropanol or 1-methoxy-2-propanol. This produced 1685 g of a 27.2% strength polyurethane urea solution in toluene/isopropanol/1-methoxy-2-propanol having a viscosity of 41000 mPas at 22° C.

### Example 5: (Comparison)

At 110° C., 400.0 g of PolyTHF 2000, 104.6 g of isophorone diisocyanate and 126.6 g of toluene were reacted to a constant NCO content of 3.6%. The mixture was allowed to cool and was diluted with 422.4 g of toluene and 377.8 g of isopropanol. At room temperature, a solution of 48.4 g of 4,4′-diaminodicyclohexylmethane dissolved in 327.5 g of 1-methoxy-2-propanol was added. After build up of the molecular weight was completed and the desired viscosity range was reached, the mixture was allowed to stand overnight in order to block the residual isocyanate content with isopropanol or 1-methoxy-2-propanol. This produced 1807 g of a 30.9% strength polyurethane urea solution in toluene/isopropanol/1-methoxy-2-propanol having a viscosity of 27800 mPas at 22° C.

### Examples 6a-e

Production of the Cell Culture Carriers by Coating with Polyurethane Solutions

The coatings were produced on glass microscope slides of the 25×25 mm size using a spin coater (RC5 Gyrset 5, Karl Süss, Garching, Germany). A microscope slide for this purpose was clamped onto the sample disk of the spin coater and homogeneously coated with approximately 0.5-1 ml of organic 5% strength polyurethane solution. All organic polyurethane solutions were diluted to a polymer content of 5% by weight with a solvent mixture of 65% by weight toluene and 35% by weight isopropanol (2:1). By rotating the sample disk for 120 sec at 3000 revolutions per minute, a homogeneous coating was obtained which was dried for 2 h at 60. The resultant polyurethane coatings were 7-sterilized at a dose of 50 kGy at room temperature for use for cell culture experiments.

TABLE 1
Coatings produced from the raw materials of examples 1-5

| Polyurethane solution used | Coating |
|---|---|
| PU solution of example 1 | Example 6a |
| PU solution of example 2 | Example 6b |
| PU solution of example 3 | Example 6c |
| PU solution of example 4 | Example 6d |
| PU solution of example 5 | Example 6e |

### Example 7

Study of Cell Growth on Native PU Surfaces

a) General Protocol for the Cell Culture of Multipotent Cell Line C2C12

Multipotent cells C2C12 mouse cells were cultured in DMEM (Dulbecco's Modified Eagle Medium) which contains 10% fetal calf serum (FBS) for 2 to 3 days at 37° C. in a moistened atmosphere containing 5% carbon dioxide. The cells were subcultured with an about 85% covering, in that the cells were flushed twice with PBS and then treated for 5 min with trypsin-EDTA in order to detach the cells from the culture surface. The cells were then taken up in DMEM, centrifuged off and plated out at a cell density of 700 cells/cm².

For the proliferation study, the cells were likewise cultured at a density of about 700 cells/ml on the native polyurethane substrates of examples 6a-e. For the control, polystyrene (Tissue Culture Polystyrene, (TCP)) and glass were colonized at the same cell densities. After 24 h at 37° C. in a moistened atmosphere containing 5% carbon dioxide, the mitochondrial respiration was determined by Alamar Blue according to the manufacturer's protocol (see J. Immunol. Methods 1997, 204, 205 for the method). For this purpose, at defined time points, 15% Alamar Blue was added to the cell culture and the culture was incubated for 4 h at 37° C. The cell culture medium was pipetted off and the optical density was measured undiluted at a wavelength of 570 and 630 nm (Tecan Genios Miroplate Reader). In order to demonstrate the reaction of Alamar Blue due to cellular respiration, the absorption ratio of metabolized Alamar Blue and unused Alamar Blue was formed. The measured optical densities were evaluated as relative units.

Fresh cell culture medium was added to the cell culture for further studies. The study of cell concentration was repeated in the abovedescribed manner each further day.

b) General Protocol for Cell Culture of Mesenchymal Cell Line C3H10T0.5

Mesenchymal mouse C3H10T0.5 stem cells were cultured in Eagle's Basal Medium which contained Glutamax and Earle's Salt, enriched with 10% fetal calf serum (FBS). The cells were kept in a moist atmosphere with addition of 5% carbon dioxide. The cells were subcultured with about 70% coverage, as described in example 7a for the multipotent C2C12 cells, and plated out at a concentration of 2×10³ cells/cm². The low plating densities were selected in order to prevent contact inhibition and the selection of cell variants.

For the proliferation study, the native polyurethane substrates of examples 6a-e were colonized by the cells at a density of about 700 cells/cm². For the control, polystyrene (Tissue Culture Polystyrene, (TCP)) and glass were colonized at the same cell densities. After 24 h at 37° C., the mitochondrial respiration was determined by Alamar Blue according to the manufacture's protocol as described in example 7a.

c) Results with C2C12

TABLE 2
Growth of C2C12 cells on the coatings produced (relative units)

| Coating | Day 1 | Day 3 | Day 4 | Day 5 | Day 6 | Day 7 |
|---|---|---|---|---|---|---|
| Tissue Culture | 18 | 48 | 67 | 145 | 221 | 183 |
| Polystyrene | | | | | | |
| (TCP) | | | | | | |
| Example 6a | 20 | 44 | 53 | 122 | 150 | 184 |
| Example 6b | 21 | 41 | 64 | 129 | 182 | 180 |
| Example 6c | 21 | 44 | 53 | 136 | 164 | 187 |
| Example 6e | 21 | 36 | 29 | 27 | 30 | 35 |

The coatings according to the invention of examples 6a, 6b and 6c permit growth which is comparable to the growth on the previously conventional tissue culture polystyrenes. The comparative coating of example 6e, in contrast, does not permit growth of the cell lines and is therefore unusable as a culture carrier.

d) Results with C3H10T0.5

TABLE 3
Growth of the C3H10T0.5 cells on the coatings produced (relative units)

| | Day | | Day | | Day | | |
|---|---|---|---|---|---|---|---|
| Coating | 1 | Day 3 | 4 | Day 5 | 6 | Day 7 | Day 8 |
| TCP | 26 | 36 | 42 | 99 | 140 | 173 | 156 |
| Example 6a | 26 | 38 | 43 | 83 | 114 | 133 | 149 |
| Example 6b | 27 | 36 | 38 | 70 | 116 | 133 | 159 |
| Example 6c | 27 | 37 | 38 | 77 | 122 | 159 | 169 |
| Example 6e | 25 | 22 | 17 | 18 | 24 | 21 | 27 |

The coatings of examples 6a, 6b and 6c permit growth which is comparable with the growth on the previously conventional Tissue Culture Polystyrenes. The comparative coating in example 6e, in contrast, does not allow growth of the cell lines and is therefore unusable as a culture carrier.

In a further experimental series, the growth of the C3H10T0.5 cells on the 6d coating is compared with the cell growth on Tissue Culture Polystyrene.

TABLE 4
Growth of C3H10T0.5 cells on the inventive coating of example
6d (relative units)

| | Day | Day | Day | Day | Day | Day | Day | |
|---|---|---|---|---|---|---|---|---|
| Coating | 1 | 2 | 3 | 4 | 6 | 7 | 8 | Day 9 |
| TCP | 3.7 | 3.7 | 8.0 | 9.8 | 39.1 | 93.1 | 144.5 | 160.4 |
| Example 6d | 3.3 | 2.5 | 4.2 | 8.8 | 42.2 | 49.5 | 108.3 | 148.0 |

The coating according to the invention of example 6d permits growth which is comparable to the growth on the previously conventional Tissue Culture Polystyrene.

The growth experiments of tables 3 and 4 were carried out on different days. Owing to the significant width of variation of experimental series in cell biology, each of these experiments requires an internal standard, here Tissue Culture Polystyrene (TCP). The absolute values of the growth curves between different experiments cannot be compared directly with one another. However, the relation of both experiments to the internal standard makes it quite clear that the growth gives results comparable to the standard TCP both on the coatings of examples 6a, 6b and 6c and on the coating of example 6d.

### Example 8

Cell Growth of the C3H10T0.5 Cell Line Without Differentiation

a) General Protocol:

Mesenchymal C3H10T0.5 mouse stem cells were cultured in Eagle's Basal Medium, which contains Glutamax and Earle's Salt, enriched with 10% fetal calf serum (FBS). The cells were kept in a moist atmosphere with addition of 5% carbon dioxide. The cells were subcultured with about 70% coverage and plated out at a concentration of 2×10³ cells/cm². These low plating densities were selected in order to prevent contact inhibition and the selection of cell variants.

For studying the capacity of the polyurethane coatings for inhibiting osteogenic differentiation, the cells were cultured in the presence of BMP-2 (Bone Morphogenetic Protein). The conditions are the same as described in example 7b, only that the BMP-2 is additionally added to the cell culture medium. For promotion of mineralization, in addition, 200 μM ascorbic acid and 10 mM glycerol phosphate were further added. BMP-2 is a known agent for inducing osteogenic differentiation. For this purpose, C3H10T0.5 mesenchymal stem cells were plated out at a concentration of 1.25×10⁴ cells/cm² in full medium with addition of 500 ng/ml of BMP-2 onto a native polyurethane coating of example 6d. As control, the same cell cultures were seeded onto TCP and glass under identical experimental conditions. During differentiation of the stem cells to form osteoblasts, the content of the enzyme alkaline leukocyte phosphatase (ALP) was used as marker for the extent of the differentiation. For determining the ALP, cells were withdrawn from the culture, washed with PBS and lysed by freezing with addition of 1% by volume of Triton X-100.

The reagent for determining alkaline leukocyte phosphatase was produced by adding 5 ml of 16 mM p-nitrophenol phosphate and 20 μl of a 1 M aqueous MgCl₂ solution to 5 ml sodium borate-sodium hydroxide buffer with a pH of 9.8.

At 37° C., 50 μl of cell lysate were incubated with 200 μl of reagent of the abovementioned composition and the color formation reaction was followed continuously at 410 nm. The activity of ALP was standardized to a total protein content with a BCA Assay from Pierce, in order to obtain specific ALP activities with the unit mmol/min/mg of protein. For control, undifferentiated cells were studied for ALP activity at a density of 1.25×10⁴ cells/cm² as described.

b) Results

TABLE 5
Effect of the coating of example 6d on prevention of the differentiation
of the mesenchymal C3H10T0.5 stem cells in control medium
without BMP-2 as inducing agent (values are activity of the alkaline
leukocyte phosphatase in nmol/min/mg of protein)

| Surface | ALP activity (nmol/min/mg protein) |
|---|---|
| Tissue Culture Polystyrene | 0.83 |
| Glass | 0.54 |
| Polyurethane film of example 6d | 0.20 |

The activity of alkaline leukocyte phosphatase is markedly lower after cell culture on the polyurethane film of example 6d than the activity after culture on Tissue Culture Polystyrene or on glass. In the in-vitro culturing, on the polyurethane film, less premature differentiation of the cells occurs, which, compared with the previously customary materials such as Tissue Culture Polystyrene or glass, is advantageous.

TABLE 6
Effect of the coating of example 6d on prevention of differentiation
of the mesenchymal C3H10T0.5 stem cells in control medium
with addition of BMP-2 as inducing agent of osteogenic
differentiation (values are activity of alkaline
phosphatase in nmol/min/mg of protein)

| Surface | ALP activity (nmol/min/mg protein) |
|---|---|
| Tissue Culture Polystyrene | 3.41 |
| Glass | 1.60 |
| Polyurethane film of example 6d | 0.20 |

10

The activity of ALP is markedly lower after cell culture on the polyurethane film of example 6d than the activity after culture on Tissue Culture Polystyrene or on glass. By adding the inducing agent BMP-2, the differentiation of the mesenchymal cell culture increases markedly on the previously conventional cell culture carriers polystyrene or glass (see tables 5 and 6 for comparable values). On the polyurethane film according to the invention, during in-vitro culturing, despite the presence of an inducing agent, no elevated premature differentiation of cells occurs.

### Example 9

Cell Growth of Human Mesenchymal Stem Cells Without Differentiation on Native PU Coating of Example 6d

a) General Preparation of the Human Stem Cells

Human mesenchymal stem cells were isolated from bone marrow of healthy donors according to the protocol of Oswald et al., Stem Cells 2004, 22, 377-384. The cells were taken from a donor by puncture from the iliac crest. From the puncture cell mixture, mononuclear cells were isolated by removing the erythrocytes by density gradient centrifugation. The mononuclear cells are placed in a cell culture bottle in such a manner that the mesenchymal stem cells adhere to the substrate. The cell culture consists of DMEM with a content of 1 g/l of glucose and 10% by volume of fetal calf serum. The cells were cultured at 37° C. in an atmosphere saturated with water vapor having a 5% content of carbon dioxide. The culture time is dependent on donor, and was 2 weeks in the case described.

The residual erythrocytes are washed down after 72 h. The remaining mesenchymal stem cells multiply in the cell culture. After harvesting, the cells are characterized phenotypically and the differentiation behavior determined.

The cells were subcultured at a degree of covering of about 80% for a maximum of five passages. The resultant cells were plated out onto the polyurethane coating of example 6d at a cell density of 1×10⁴ cells/cm². For the control, the same cell cultures were plated out onto TCP and onto glass.

The human mesenchymal stem cells were cultured as in example 7a.

The osteogenic differentiation was studied in DMEM with addition of 10% by volume FBS and also human recombinant BMP-2 (200 ng/ml) with further addition of 200 μM ascorbic acid and 10 mM glycerol phosphate by seeding onto polyurethane films of example 6d. The method is the same as in example 8, except that here, in the case of human mesenchymal stem cells the cell culture was further run on the fourth day without adding fetal calf serum. As controls, the cells were seeded onto TCP and onto glass. On day 4, the medium was changed to DMEM which contained ITS (medium addition of: insulin 6.25 mg/mil; transferrin 6.25 mg/ml; selenous acid 6.25 μg/ml; bovine serum albumin 0.125 g/ml and linoleic acid 5.35 mg/ml) and to which fresh BMP-2 was added. The activity of alkaline leukocyte phosphatase for evaluating osteogenic differentiation were determined in accordance with the protocol of example 8.

In addition, as a second marker for differentiation of the cells, matrix mineralization of the cells was determined by staining with Alizarin S as in J. Jadlowiec et al., J. Biol. Chem. 2004, 279, 53323-53330. For this purpose, the cells were withdrawn at certain time points, washed with 0.5 ml PBS, and fixed at −20° C. for 1 h with 0.5 ml of ethanol (70% by weight in water). The cells were then washed with 0.5 ml of twice-distilled water and stained with 0.5 ml of an aqueous 40 nM alizarin solution adjusted to a pH of 4.2 with ammonia. Excess alizarin was removed by washing with water. The dye bound in the matrix of the osteoblasts formed was dissolved by incubating the strained cells for two hours at room temperature in 300 μl of a 10% strength by weight aqueous hexadecylpyridinium chloride solution and the optical density of this solution was determined at 570 nm.

b) Results for Human Mesenchymal Stem Cells

TABLE 7
Effect of the coating of example 6d on preventing the differentiation of
human mesenchymal stem cells in control medium without BMP-2
as inducing agent (values are activity of the alkaline leukocyte
phosphatase in nmol/min/mg of protein)

| Surface | ALP activity (nmol/min/mg protein) |
|---|---|
| Tissue Culture Polystyrene | 8.92 |
| Glass | 9.85 |
| Polyurethane film of example 6d | 2.36 |

The activity of alkaline leukocyte phosphatase is, after cell culture on the polyurethane film of example 6d, markedly lower than the activity after culture of the same cells on Tissue Culture Polystyrene or on glass. In the case of in-vitro culture, less premature differentiation of the cells occurred on the polyurethane film, which, compared with the previously customary materials such as Tissue Culture Polystyrene or glass, is advantageous.

TABLE 8
Effect of the coating of example 6d on preventing the differentiation of
human mesenchymal stem cells in control medium with addition of
BMP-2 as inducing agent (values are activity of alkaline phosphatase
in nmol/min/mg protein)

| Surface | ALP activity (nmol/min/mg protein) |
|---|---|
| Tissue Culture Polystyrene | 41.28 |
| Glass | 15.43 |
| Polyurethane film of example 6d | 1.48 |

When BMP-2 is added as inducing agent, in the conventional cell culture carriers polystyrene and glass, a marked premature osteogenic differentiation of the stem cells is observed. The polyurethane film according to the invention, in contrast, displays only a very slight unwanted premature differentiation. The absolute value of the activity of alkaline phosphatase is not higher than that without addition of BMP-2 (see table 7).

TABLE 9
Effect of the coating of example 6d on the prevention of osteogenic
differentiation of human mesenchymal stem cells in control medium
with addition of BMP-2 as inducing agent (values are optical
densities of the alizarin determination)

| Surface | Alizarin (absorption at 570 nm)) |
|---|---|
| Tissue Culture Polystyrene | 1.54 |
| Glass | 1.06 |
| Polyurethane film of example 6d | 0.39 |

The staining with alizarin, just as does the detection of the activity of alkaline leukocyte phosphatase, shows that in the presence of the inducing agent BMP-2, the polyurethane film according to the invention as cell culture medium gives rise to substantially less spontaneous, premature and unwanted differentiation of the human mesenchymal stem cells than the conventional cell culture carriers polystyrene and glass.

The stein cells that multiplied on the cell culture carrier according to the invention, after the removal from the carrier, still exhibit the necessary differentiation potential and can be used correspondingly, for example for tissue engineering.

1-13. (canceled)

14. A method for producing a coated cell culture carrier which comprises applying a solution comprising a polyurethane-urea to a cell carrier and drying, wherein the polyurethane urea is obtained by reacting components comprising a polycarbonate polyol, a polyisocyanate, and a diamine.

15. The method as claimed in claim 14, wherein the diamine component comprises at least one hydroxyl group.

16. The method as claimed in claim 14, wherein the polycarbonate polyol component has a number-average hydroxyl functionality of from 1.7 to 2.3.

17. The method as claimed in claim 14, wherein the polycarbonate polyol component has a number-average hydroxyl functionality of from 1.9 to 2.1.

18. The method as claimed in claim 14, wherein the polycarbonate polyol component has a number-average molecular weight of from 400 to 6,000 g/mol.

19. The method as claimed in claim 14, wherein the polycarbonate polyol component has a number-average molecular weight of from 600 to 3,000 g/mol.

20. The method as claimed in claim 14, wherein the polyurethane urea has a number-average molecular weight of from 1,000 to 200,000 g/mol.

21. The method as claimed in claim 14, wherein the polyurethane urea has a number-average molecular weight of from 5,000 to 100,000 g/mol.

22. The method as claimed in claim 14, wherein the component used to obtain the polyurethane urea further comprise a further polyol component.

23. The method as claimed in claim 22, wherein the further polyol component is present in an amount of from 0.05 to 2 mol, based on 1 mol of the polycarbonate component.

24. The method as claimed in claim 22, wherein the further polyol component is present in an amount of from 0.1 to 1 mol, based on 1 mol of the polycarbonate component.

25. The method as claimed in claim 22, wherein the further polyol component has a number-average molecular weight of from 62 to 500 g/mol.

26. The method as claimed in claim 22, wherein the further polyol component has a number-average molecular weight of from 62 to 200 g/mol.

27. The method as claimed in claim 14, wherein the diamine component is present in an amount of from 0.1 to 3 mol, based on 1 mol of the polycarbonate component.

28. The method as claimed in claim 14, wherein the diamine component is present in an amount of from 0.3 to 2.5 mol, based on 1 mol of the polycarbonate component.

29. The method as claimed in claim 14, wherein the polyisocyanate component is present in an amount of from 1.0 to 5 mol, based on 1 mol of the polycarbonate component.

30. The method as claimed in claim 14, wherein the polyisocyanate component is present in an amount of from 1.0 to 4.0 mol, based on 1 mol of the polycarbonate component.

31. The method as claimed in claim 14, wherein the solution further comprises proteins.

32. The method as claimed in claim 31, wherein the solution further comprises proteins of the extracellular matrix.

33. A coated cell culture carrier obtained by the method according to claim 14.

34. A method for in-vitro culturing of stem cells comprising applying a stem cell to the coated cell culture carrier as claimed in claim 14.

35. A method for in-vitro culturing of mesenchymal stem cells comprising applying a mesenchymal stem cell to the coated cell culture carrier as claimed in claim 14.
